(19) United States
(12) Patent Application Publication
(10) Pub. No.: US 2025/0098971 A1
(43) Pub. Date: Mar. 27, 2025

(54) INTRAUTERINE BALLOON FOR INDUCTION OF LABOR WITH INTRAUTERINE PRESSURE CATHETER

(71) Applicants: Jennifer M. GOLDKAMP, Saint Louis, MO (US); MEC Happens LLC, Saint Louis, MO (US)

(72) Inventor: Jennifer M. Goldkamp, Saint Louis, MO (US)

(21) Appl. No.: 18/728,820

(22) PCT Filed: Jan. 12, 2023
(86) PCT No.: PCT/US2023/010707

§ 371 Date: Jul. 12, 2024

Related U.S. Application Data
(60) Provisional application No. 63/298,909, filed on Jan. 12, 2022.

(51) Int. Cl. A61B 5/03; A61B 5/00; A61B 17/42; A61M 25/00

(52) U.S. Cl. CPC A61B 5/035; A61B 5/4343; A61B 5/6853; A61B 17/42; A61M 25/0026; A61B 2017/4225

(57) ABSTRACT

Disclosed herein are devices, systems, and methods related to monitoring intrauterine pressure. An example method may include inserting a catheter assembly into an uterus of a patient such that a sensor of the catheter assembly is placeable within the uterus and an expandable region is positioned to facilitate dilation of a cervix, the sensor is operable such that an intrauterine pressure measurement is obtainable while the expandable region is expanded. Also, the monitoring intrauterine pressure may include expanding the expandable region to facilitate dilating the cervix. Furthermore, the monitoring intrauterine pressure may include obtaining an intrauterine pressure measurement of an amniotic space of the uterus.

### FIELD

The present disclosure relates generally to apparatuses, systems, and methods regarding the combination of an intrauterine balloon and intrauterine pressure catheter for the purpose of simultaneous cervical ripening for induction of labor and monitoring contractions through intrauterine pressure changes. More specifically, the disclosure relates to apparatuses, systems, and methods.

### BACKGROUND

Induction of labor (IOL) is undertaken in approximately one out of four pregnancies in the United States. Many options for management of IOL exist, including promoting maternal cervical dilation through medication administration and through applying physical pressure to the cervix, also known as mechanical dilation. Two mechanical dilation methods to induce early cervical dilation are commonly utilized; laminaria (dried seaweed or kelp) and placement of an intra-uterine balloon.

A bladder catheter (Foley catheter) has traditionally been used for manual dilation of the maternal cervix due to availability and ease of use. The leading portion of the catheter is placed transvaginally through the cervix until the balloon portion of the catheter is just above the cervix at the level of the internal cervical os, in the intrauterine cavity, the balloon is then inflated with fluid. The balloon sits on the internal cervical os and places continuous pressure against the cervix, leading to dilation and eventual expulsion of the balloon.

As a part of monitoring labor, many obstetric providers choose to place an intra-uterine pressure catheter/monitor (IUPC). An IUPC is placed transvaginally through the cervix and advanced into the uterine cavity where it remains as long as the obstetric provider deems necessary. In general, an IUPC can be placed when the cervix is 0.5 cm to 10 cm dilated. The IUPC measures the frequency and strength of uterine contractions through a connection to an external monitor/tocometer. In certain subsets of women, monitoring the strength of a contraction is of higher importance in order to maintain a safe environment for the mother and the fetus. The largest group of women that this applies to is those undergoing a trial of labor after cesarean (TOLAC). These women are limited in medications options for labor induction and are at higher risk for complications related to labor and delivery, specifically uterine rupture. In addition, correlation of contractions in relationship to the fetal heart rate monitoring allows for modification of maternal characteristics such as position, and medication titration, to promote a successful and healthy vaginal delivery. External uterine contraction monitoring is an additional option for monitoring contractions, however strength of the contraction cannot be determined from the external monitoring, and external monitoring is limited by some maternal characteristics such as obesity.

Due to the location and size of an intrauterine balloon utilized for induction of labor, co-placement of additional intra-uterine device is not feasible. This poses a challenging medical quandary when use of two devices is indicated, particularly the addition of an IUPC, given the latter device can help guide clinical management.

### SUMMARY

The combination of an intra-uterine balloon for induction of labor and an intra-uterine pressure monitor has not been previously described. This would allow for obstetric providers to monitor the intra-uterine pressure while the patient is undergoing an induction of labor with a mechanical cervical dilator in place. This device could be used in any patient undergoing induction of labor, and is of particular use in women under TOLAC due to desire to monitor intrauterine pressure and limitations of induction methods in this subgroup of pregnant women.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

Some implementations herein relate to devices and systems including such devices. For example, intrauterine catheter assembly may include a catheter that is axially elongate and has a tube wall that defines a lumen disposed in a catheter shaft of the catheter, the tube further has an expandable region that is positioned along the shaft at the tube wall and is outwardly expandable to facilitate dilation of a cervix. Intrauterine catheter assembly may also include a sensor positioned along the shaft at a position that is distal to the position of the expandable region, the sensor is operable such that an intrauterine pressure measurement is obtainable while the expandable region is expanded. Other embodiments of this aspect include corresponding computer systems, apparatus, and computer programs recorded on one or more computer storage devices, each configured to perform the actions of the methods.

The described implementations may also include one or more of the following features. Intrauterine catheter assembly where the position of the sensor is such that the sensor is placeable in a uterus with the expandable region positioned in the lower uterine segment. Intrauterine catheter assembly where the position of the sensor is such that the sensor is placeable beyond the lower uterine segment. Intrauterine catheter assembly may include a labor monitor that is operatively connected to the tube and the sensor, the labor monitor is configured to perform at least one of: filling the expandable region; evacuating the expandable region; receiving information that is indicative of the intrauterine pressure measurement; delivering fluid within the amniotic space; and evacuating fluid from the amniotic space. Intrauterine catheter assembly may include a pressure catheter that is receivable within the lumen, and where the sensor is provided by the pressure catheter. Intrauterine catheter assembly where the lumen is a first lumen and where the tube further may include a second lumen, the first lumen terminates at and is in fluid communication with a position along the shaft that is distal to the expandable region, and the second lumen terminates at and is in fluid communication with the expandable region such that the second lumen facilitates at least one of filling and evacuating the expandable region. Intrauterine catheter assembly where the first lumen terminates at one or more perforations disposed at a distal portion of the catheter such that included tubes that the first lumen facilitates delivery of fluids within an amniotic space of a uterus. Intrauterine catheter assembly may include at least two fill ports, a first fill port is in communication with the first lumen to facilitate obtaining the intrauterine pressure measurement and a second fill port is in communication with the second lumen to facilitate filling the expandable region. Intrauterine catheter assembly may include either (i) a third fill port in communication with the second lumen to facilitate fluid delivery to delivery of fluids within an amniotic space or (ii) a socket in communication with the second lumen to facilitate fluid delivery to delivery of fluids within the amniotic space. Intrauterine catheter assembly may include a central axis extending through the tube and where a distal portion of the tube is angled relative to the central axis upon inflation of the expandable region. Implementations of the described techniques may include hardware, a method or process, or a computer tangible medium.

Some implementations herein relate to a method. For example, method may include inserting a catheter assembly into a uterus of a patient such that a sensor of the catheter assembly is placeable within the uterus and an expandable region is positioned to facilitate dilation of a cervix, the sensor is operable such that an intrauterine pressure measurement is obtainable while the expandable region is expanded. Method may also include expanding the expandable region to facilitate dilating the cervix. Method may furthermore include obtaining an intrauterine pressure measurement of an amniotic space of the uterus. Other embodiments of this aspect include corresponding computer systems, apparatus, and computer programs recorded on one or more computer storage devices, each configured to perform the actions of the methods.

The described implementations may also include one or more of the following features. Method may include adjusting the pressure of the amniotic space by performing at least one of delivering fluid within the amniotic space and evacuating fluid from the amniotic space. Method may include adjusting the position of the sensor within the uterus relative to the expandable region. Method may include adjusting an expansion of the expandable region by performing at least one of filling the expandable region and evacuating the expandable region. Method may include receiving information that is indicative of the intrauterine pressure measurement and, in response thereto, performing at least one of adjusting the pressure of the amniotic space, adjusting the position of the sensor within the uterus relative to the expandable region, and adjusting an expansion of the expandable region. Implementations of the described techniques may include hardware, a method or process, or a computer tangible medium.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a partial schematic drawing of a human reproductive system.

FIG. 2 is a plan view of two catheter configurations according to principles of the present disclosure.

FIG. 3 is a cross-section plan view taken at Sections A and B in FIG. 2.

FIG. 4 is a diagram of various operational stages of the catheter, according to principles of the present disclosure.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

In general, devices, systems, and methods disclosed herein are useful for women who would benefit from intrauterine pressure catheter monitoring for labor and benefit from concomitant use of a mechanical cervical dilation for induction of labor. Traditionally, such applications required two or more separate devices, and there is not sufficient space for an intrauterine pressure monitor if a mechanical dilator is present. Also traditionally, two balloons, one internal to the cervix and another external to the cervix, are inflated to dilate the cervix, but these applications too suffer from similar drawbacks as other traditional catheters. This disclosure provides a single device that can complete both intrauterine pressure catheter monitoring and mechanical cervical dilation without need of repositioning and/or removing the device. This intrauterine pressure catheter and a mechanical balloon dilator can be used for monitoring contractions and assisting with induction of labor, respectively.

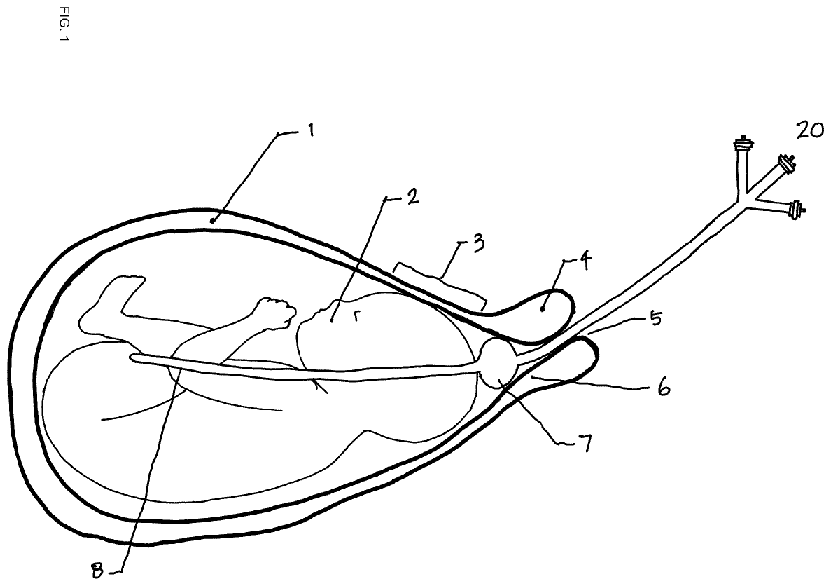

FIG. 1 shows an embodiment in which principles of the present disclosure can be used. In particular, FIG. 1 shows a schematic diagram of a human reproductive system during pregnancy. As illustrated, this system includes a uterus 1, a cervix 4 defining a cervical canal, and a fetus 2 contained within the uterus 1. As is known in the art, the cervical opening into the vagina may be referred to as the external cervical os 5; the cavity running the length of the cervix 4 is the endocervical canal; the opening of the endocervical canal into the uterine cavity may be referred to as the internal cervical os 6.

Inserted into the uterus 1 through the cervical canal is a multi-lumen catheter 20 with which to measure intrauterine pressure. As discussed elsewhere herein, the catheter 20 can be integrated into a catheter assembly that comprises a catheter 20 and a pressure monitor 12. In general, the intrauterine portion of the catheter 20 includes a proximal end and a distal end. The distal end can be defined by or otherwise include an intrauterine portion of the catheter 20 such that the distal end has a proximal intrauterine portion and a distal intrauterine portion. The proximal intrauterine portion includes an expandable segment 7 (e.g., a balloon or other inflatable) that can be positioned above the cervical canal as shown and inflated during operation to apply pressure against the cervical canal to progressively dilate the cervix 4. The distal intrauterine portion includes a pressure monitor 8 that is positioned beyond the lower uterine segment 3. It is contemplated that in some instances, the pressure monitor 12 is positioned within the lower uterine segment 3. Also included are through holes that allow fluid to be directed into the uterus 1. The external portion of the catheter 20 (e.g., the portion that is not the intrauterine portion of the catheter 20) can be in fluid communication with the distal uterine portion via one or more lumens of the catheter 20, where each lumen is in fluid communication with a fill port and extends through the catheter 20 to the distal end. It should be noted that although a certain quantities of components (e.g., balloons, pressure monitors, apertures 13) are depicted, one skilled in the art would appreciate that these quantities can be varied (e.g., 1 to 2, 1 to 3, 2 to 5,and the like) without departing from the scope of this disclosure.

As shown, the catheter 20 includes at least two fill ports, one of which (e.g., a pressure monitor port 10) can be used to facilitate sensing pressure via the pressure monitor 12. Another one of the fill ports (e.g., a balloon fill port 9) can be used to facilitate operation (e.g., inflation and/or deflation) of the balloon. Where there are at least three fill ports, yet another one of the fill ports (e.g., a fluid introduction port 11) can be used to facilitate injecting fluid (e.g., lactated ringers or saline) from a fluid source through the distal end and into the uterus 1. More details about the catheter 20 are discussed below. In operation, the balloon can be placed in an intrauterine cavity at the level of the internal cervical os 6. At the same time, the pressure monitor 12 can be placed in the intrauterine cavity, and would extend past the lower uterine segment 3. The balloon can be inflated, e.g., via a balloon fill lumen 15 that extends from the balloon fill port 9 and terminates at the balloon. As contractions occur, the fetus 2 can press against the balloon, forcing the balloon further down the cervical canal and thereby further dilating the cervix 4. The intrauterine cavity can be filled with fluid from the fluid introduction port 11 via apertures 13 that are in fluid communication with the fluid introduction port 11 via a fluid introduction lumen 47. The apertures 13 can be positioned at a tip of the distal end as shown.

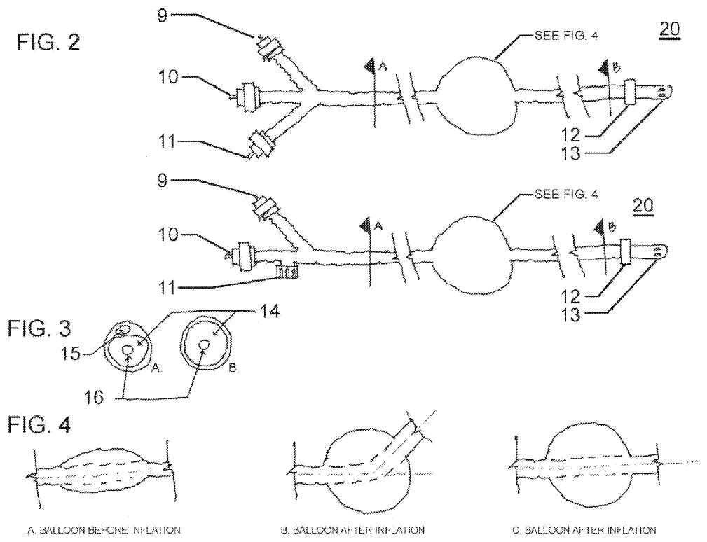

FIGS. 2-4 show various features of catheters disclosed herein. In particular, FIG. 2 shows two configurations of the catheter 20: Configuration A where the catheter 20 includes three fill ports and Configuration B where the catheter 20 includes two fill ports and a capped socket 11 (e.g., a fluid introducer) that is connectible to a fluid source. FIG. 3 shows cross sections A and B in FIG. 1. And FIG. 4 shows various stages (e.g., Stages A, B, and C.) of the balloon during operation. In particular, Stage A shows the balloon before inflation, and both Stages B and C show the balloon after inflation where Stage B has a designed-in angle after inflation and Stage C does not include the designed-in angle.

In more detail, FIG. 2 shows the device in its entirety. The portion of the device that is intended for external use has three ports and is shown as two different configurations. The three ports represent access to the supra-cervical balloon, the intrauterine pressure monitor 12, and the fluid management. The pressure monitor 12 attaches to an external cable that is connected to a labor monitor. As such, examples disclosed herein include such monitors, related systems, and programs such as non-transitory computer readable mediums with instructions for performing functions of the labor monitor. The intrauterine portion of the device begins with the cervical dilator balloon and a long tube for intrauterine pressure monitoring and management of intrauterine fluids. The pressure monitor 12 can include one or more pressure sensors. pressure monitor 12 wiring connects from the most proximal end of the device into the labor monitor machine. As illustrated, this wiring runs the length of the device to the end and terminates at a small area below the perforations for the pressure monitor 12.

At least one or each of these pressure sensors can be provided by a pressure catheter 16. There can be a small number of electronics/materials that goes through the pressure catheter 16 lumen, which is protected from the surrounding fluid if the pressure catheter 16 is routed through the fluid delivery lumen of the catheter 20. In addition, or in alternative, others of these pressure sensors or all of these pressure sensors may provided by the catheter 20. For instance, under these circumstances, the catheter 20 can include an internally and/or externally mounted pressure sensor. If required, wires for these pressure sensors may be strung through a primary or secondary lumen of the catheter 20. In examples, there ma be a third lumen dedicated to these wires.

FIG. 3 is a cross section of the tube below the cervical dilator balloon (Section A) and above the balloon (Section B). Section A has a tube for inflation or deflation of the balloon dilator and for the pressure monitor 12, the remaining space is for fluid infusion. Above the level of the balloon seen at Section B, the tube for the pressure monitor 12 and the outer tube for fluid management is present.

FIG. 4 demonstrates the cervical balloon dilator. The balloon will be accessed through an external port as demonstrated in FIG. 1. The balloon will be inserted deflated (Stage A of FIG. 4) and then filled once intrauterine placement is confirmed by cervical examination. The catheter 20 can optionally include a bend that is accentuated during inflation such that a central axis of the catheter 20 at the distal portion of the catheter 20 is transversely positioned relative to the central axis of the catheter 20 that is immediately proximal to the distal portion of the catheter 20 that has the bend (Stage B in FIG. 4). In examples, this transverse positioning can result in an approximate 45-degree angle bend that can help circumvent the fetus 2, for example. This bend can be caused, for example, by designed-in features of the catheter 20. For instance, a portion of the catheter 20 can be imbibed or otherwise stiffer than an opposing portion of the catheter 20 such that inflation causes the stiffer portion of the catheter 20 to remain generally stationary relative to the less stiff opposing side. In certain instances, the catheter 20 may have an external stiffening portion (e.g., a knob or sleeve) with which to cause the angle bend. These examples are just some of many examples.

As noted elsewhere herein, without reference to any particular figure, disclosed herein is a system of one or more computers that can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, non-transitory computer-readable medium may include one or more instructions that, when executed by one or more processors of a device, cause the device to: insert a catheter assembly into an uterus of a patient such that a sensor of the catheter assembly is placeable within the uterus and an expandable region is positioned to facilitate dilation of a cervix, the sensor is operable such that an intrauterine pressure measurement is obtainable while the expandable region is expanded. These instructions may furthermore include expanding the expandable region to facilitate dilating the cervix. These instructions may furthermore include obtaining an intrauterine pressure measurement of an amniotic space of the uterus. Other embodiments of this aspect include corresponding computer systems, apparatus, and computer programs recorded on one or more computer storage devices, each configured to perform the actions of the methods.

Implementations may include one or more of the following features. Non-transitory computer-readable medium may include adjusting the pressure of the amniotic space by performing at least one of delivering fluid within the amniotic space and evacuating fluid from the amniotic space. The instructions may include adjusting the position of the sensor within the uterus relative to the expandable region. The instructions may include adjusting an expansion of the expandable region by performing at least one of filling the expandable region and evacuating the expandable region. The instructions may include receiving information that is indicative of the intrauterine pressure measurement and, in response thereto, performing at least one of adjusting the pressure of the amniotic space, adjusting the position of the sensor within the uterus relative to the expandable region, and adjusting an expansion of the expandable region. Implementations of the described techniques may include hardware, a method or process, or a computer tangible medium.

This disclosure has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps can be added or omitted without departing from the scope of this disclosure. Such steps can include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections can be present in a practical system. However, the benefits, advantages, solutions to problems, and any elements that can cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone can be present in an embodiment, B alone can be present in an embodiment, C alone can be present in an embodiment, or that any combination of the elements A, B or C can be present in a single embodiment; for example, A and B, A and C, B and C, or A and B and C.

In the detailed description herein, references to “one embodiment,” “an embodiment,” “an example embodiment,” etc., indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112 (f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

While the present disclosure has been described as having an exemplary design, the present invention can be further modified within the spirit and scope of this disclosure. This application is therefore intended to cover any variations, uses, or adaptations of the invention using its general principles. Further, this application is intended to cover such departures from the present disclosure as come within known or customary practices in the art to which this invention pertains.

1. An intrauterine catheter assembly comprises:
a catheter that is axially elongate and has a tube wall that defines a lumen disposed in a catheter shaft of the catheter, the tube further has an expandable region that is positioned along the shaft at the tube wall and is outwardly expandable to facilitate dilation of a cervix, and
a sensor positioned along the shaft at a position that is distal to the position of the expandable region, the sensor is operable such that an intrauterine pressure measurement is obtainable while the expandable region is expanded.

2. The intrauterine catheter assembly of claim 1, wherein the position of the sensor is such that the sensor is placeable in a uterus with the expandable region positioned in the lower uterine segment.

3. The intrauterine catheter assembly of claim 2, wherein the position of the sensor is such that the sensor is placeable beyond the lower uterine segment.

4. The intrauterine catheter assembly of claim 1, further comprising a pressure catheter that is receivable within the lumen, and wherein the sensor is provided by the pressure catheter.

5. The intrauterine catheter assembly of claim 1, wherein the lumen is a first lumen and wherein the tube further comprises a second lumen, the first lumen terminates at and is in fluid communication with a position along the shaft that is distal to the expandable region, and the second lumen terminates at and is in fluid communication with the expandable region such that the second lumen facilitates at least one of filling and evacuating the expandable region.

6. The intrauterine catheter assembly of claim 5, wherein the first lumen terminates at one or more perforations disposed at a distal portion of the catheter such that the first lumen facilitates delivery of fluids within an amniotic space of a uterus.

7. The intrauterine catheter assembly of claim 5, further comprising at least two fill ports, a first fill port is in communication with the first lumen to facilitate obtaining the intrauterine pressure measurement and a second fill port is in communication with the second lumen to facilitate filling the expandable region.

8. The intrauterine catheter assembly of claim 7, further comprising either (i) a third fill port in communication with the second lumen to facilitate fluid delivery to delivery of fluids within an amniotic space or (ii) a socket in communication with the second lumen to facilitate fluid delivery to delivery of fluids within the amniotic space.

9. The intrauterine catheter assembly of claim 1, further comprising a central axis extending through the tube and wherein a distal portion of the tube is angled relative to the central axis upon inflation of the expandable region.

10. The intrauterine catheter assembly of claim 1, further comprising a labor monitor that is operatively connected to the tube and the sensor, the labor monitor is configured to perform at least one of:
filling the expandable region;
evacuating the expandable region;
receiving information that is indicative of the intrauterine pressure measurement;
delivering fluid within the amniotic space; and
evacuating fluid from the amniotic space.

11. A method of monitoring intrauterine pressure, the method comprising:
inserting a catheter assembly into a uterus of a patient such that a sensor of the catheter assembly is placeable within the uterus and an expandable region is positioned to facilitate dilation of a cervix, the sensor is operable such that an intrauterine pressure measurement is obtainable while the expandable region is expanded;
expanding the expandable region to facilitate dilating the cervix; and
obtaining an intrauterine pressure measurement of an amniotic space of the uterus.

12. The method of claim 11, further comprising adjusting the pressure of the amniotic space by performing at least one of delivering fluid within the amniotic space and evacuating fluid from the amniotic space.

13. The method of claim 11, further comprising adjusting the position of the sensor within the uterus relative to the expandable region.

14. The method of claim 11, further comprising adjusting an expansion of the expandable region by performing at least one of filling the expandable region and evacuating the expandable region.

15. The method of claim 11, further comprising receiving information that is indicative of the intrauterine pressure measurement and, in response thereto, performing at least one of adjusting the pressure of the amniotic space, adjusting the position of the sensor within the uterus relative to the expandable region, and adjusting an expansion of the expandable region.

16. The method of claim 12, further comprising adjusting the position of the sensor within the uterus relative to the expandable region and adjusting an expansion of the expandable region by performing at least one of filling the expandable region and evacuating the expandable region.

17. The method of claim 12, wherein adjusting the pressure of the amniotic space by performing at least one of delivering fluid within the amniotic space and evacuating fluid from the amniotic space includes adjusting the pressure of the amniotic space by performing both of delivering fluid within the amniotic space and evacuating fluid from the amniotic space.

18. The intrauterine catheter assembly of claim 3, wherein the sensor is affixed to an exterior of the tube wall with wiring for the sensor strung within the tube wall, and wherein the tube wall further includes at least one aperture formed therein for fluid management within the amniotic space, the at least one aperture being distal to the sensor.

19. The intrauterine catheter assembly of claim 4, wherein the sensor is moveable to an exterior of the tube wall, and wherein the tube wall further includes at least one aperture formed therein for fluid management within the amniotic space, the at least one aperture being distal to the sensor.

20. The intrauterine catheter assembly of claim 4, wherein the pressure catheter includes a pressure catheter lumen, and wherein the sensor is provided within the pressure catheter lumen.
